(19) United States
(12) Patent Application Publication
(10) Pub. No.: US 2024/0197678 A1
(43) Pub. Date: Jun. 20, 2024

(54) INHIBITORS OF THE PEPTIDYL-PROLYL CIS/TRANS ISOMERASE (PIN1) AND USES THEREOF

(71) Applicants: DANA-FARBER CANCER INSTITUTE, INC., Boston, MA (US); YEDA RESEARCH AND DEVLOPMENT CO. LTD., Rehovot (IL)

(72) Inventors: Inchul You, Boston, MA (US); Nathanael S. Gray, Stanford, CA (US); Tinghu Zhang, Brookline, MA (US); Sirano Dhe-Paganon, Holliston, MA (US); Nir London, Rehovot (IL); Christian Dubiella, Rehovot (IL)

(73) Assignees: DANA-FARBER CANCER INSTITUTE, INC., Boston, MA (US); YEDA RESEARCH AND DEVLOPMENT CO. LTD., Rehovot (IL)

(21) Appl. No.: 18/281,861

(22) PCT Filed: Mar. 18, 2022
(86) PCT No.: PCT/US2022/020857

§ 371 Date: Sep. 13, 2023

Related U.S. Application Data
(60) Provisional application No. 63/163,437, filed on Mar. 19, 2021.

(51) Int. Cl. A61K 31/381; A61K 31/506; A61K 31/519; C07D 333/48

(52) U.S. Cl. CPC A61K 31/381; A61K 31/506; A61K 31/519; C07D 333/48

(57) ABSTRACT

Disclosed are compounds which inhibit Pin1 activity, methods of making the compounds, pharmaceutical compositions containing the compounds, and methods of using the compounds to treat diseases or disorders characterized or mediated by dysregulated Pin1 activity.

### RELATED APPLICATIONS

This application claims the benefit of priority under 35 U.S.C. § 119(e) to U.S. Provisional Application No. 63/163,437, filed Mar. 19, 2021, which is incorporated herein by reference in its entirety.

### GOVERNMENT LICENSE RIGHTS

This invention was made with government support under grant number R01 CA205153 awarded by the National Institutes of Health. The government has certain rights in the invention.

### BACKGROUND OF THE INVENTION

Proline is unique among the amino acids because it populates both the cis and trans conformations, providing a backbone conformational switch that is controlled by prolyl isomerization. Due to the high energy barrier associated with cis to trans conversion (25-30 kcal/mol), the intrinsic isomerization process is slow (several minutes) relative to biochemical processes, and therefore catalysis by peptidyl prolyl isomerases (PPIases) is required for efficient isomerization.

Proline (Pro)-directed serine/threonine (Ser/Thr) phosphorylation (pSer/Thr-Pro) serves an essential role in cell signaling networks and is often dysregulated in cancer. Numerous oncogenes and tumor suppressors are regulated by Pro-directed phosphorylation and/or are part of signaling pathways involving such phosphorylation.

pSer/Thr-Pro reduces the intrinsically slow cis-trans isomerization process and renders the peptide bonds inaccessible for all known peptidyl-prolyl cis-trans isomerases (PPIases), except for peptidyl-prolyl cis-trans isomerase NIMA-interacting 1 (Pin1) and its homologues. Pin1 contains an N-terminal WW domain, which functions as a phosphorylated Ser/Thr-Pro binding module, and a PPIase domain, which catalyzes the cis-trans isomerization. (Zhou et al., Cell. Mol. Life Sci. 56: 788-806 (1999)).

Pin1-catalysed prolyl isomerization regulates the functions of its substrates through multiple different mechanisms, including controlling catalytic activity, turnover, phosphorylation, interactions with DNA, RNA or other proteins, and subcellular localization and processing. Pin1 often functions as a molecular timer that synchronously controls the amplitude and duration of a given cellular process. Pin1 is tightly regulated normally and its deregulation can have a major impact on the development and treatment of cancer and neurodegenerative diseases, such as Alzheimer disease. (Lu and Zhou, Nat. Rev. Mol. Cell Biol. 8:904-16 (2007)).

Pin1 is widely overexpressed and/or overactivated in cancers which correlate with poor clinical prognosis. (Lu and Hunter, Cell Res. 24:1033-49 (2014)). It has also been shown that Pin1 single nucleotide polymorphisms (SNPs) that reduce Pin1 expression are associated with a reduced risk for multiple cancers, and that Pin1-null mice are highly resistant to tumorigenesis, even after the overexpression of oncogenes or after the mutation or ablation of tumor suppressors. (Li et al., PLoS ONE 8:e68148 (2004); Wulf et al., EMBO J. 23:3397-3407 (2004); Girardini et al., Cancer Cell 20:79-91 (2011); Takahashi et al., Oncogene 26:3835-45 (2007)). Further, Pin1-null mice have been shown to develop normally to adulthood with few defects. (Lee et al., Expert Rev. Mol. Med. 13:e21 (2011)). Additionally, Pin1 overexpression disrupts cell cycle coordination and leads to chromosome instability and tumorigenesis. Pin1 activates and inactivates more than 40 oncogenes and 20 tumor suppressors, respectively. Many of these Pin1 substrates have a role in self-renewal, replicative potential and frequency of cancer stem cells (CSCs). (Zhou and Lu, Nat. Rev. Cancer 16: 463-78 (2016)). Therefore, Pin1 inhibitors may have the desirable ability to simultaneously block multiple cancer-driving pathways and CSC expansion and differentiation with limited toxicity.

### SUMMARY OF THE INVENTION

A first aspect of the present invention is directed to a compound having a structure represented by formula (PIN1-3 P1):

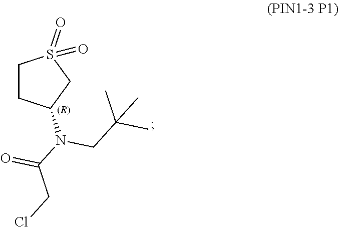

or a pharmaceutically acceptable salt thereof.

A further aspect of the present invention is directed to a compound having a structure represented by formula (PIN1-3 P2):

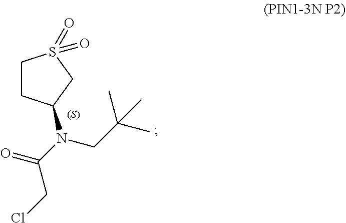

or a pharmaceutically acceptable salt thereof.

Another aspect of the present invention is directed to a pharmaceutical composition that includes a therapeutically effective amount of a compound of the invention and a pharmaceutically acceptable carrier.

A further aspect of the present invention is directed to a method for making a compound of the invention.

Another aspect of the present invention is directed to a method of treating a disease or disorder mediated by dysregulated Pin1 activity, comprising administering a therapeutically effective amount of the compound of the invention or pharmaceutically acceptable salt thereof to a subject in need thereof.

In some embodiments, the disease or disorder is cancer.

Without intending to be bound by any particular theory of operation, it is believed that compounds of the present invention exhibit their inhibitory activity by binding to at least one amino residue, e.g. cysteine 113, located in the active site of Pin1. Without intending to be bound by any theory of operation, Applicant believes that the compounds of the present invention exert their therapeutic (e.g., anti-cancer and anti-tumor) effect or benefit at least by restoring the balance of oncogene and tumor suppressor activity in tumors.

### BRIEF DESCRIPTION OF THE DRAWINGS

FIG. 1 is a chiral HPLC spectrum of Pin1-3 displaying a 1:1 ratio of enantiomers, at retention times of 1.565 and 1.976 minutes, respectively.

FIG. 2 is a chiral HPLC spectrum of Pin1-3 P1 displaying an enantiomeric excess of >99% for Pin1-3 P1, at a retention time of 1.592 minutes.

FIG. 3 is a fluorescence polarization readout following 1 h incubation of the indicated compounds with Pin1. Data points are plotted as the average of n=3 independent samples+SEM.

FIG. 4 is an immunoblot displaying the result of Molt4 cells treated in a competition format with the indicated concentrations of Pin1-3, Pin1-3 P1, and Pin1-3 P2 for 5 h, followed by cell lysis, incubation with TM-03-163 (Pin1-3 desthiobiotin), streptavidin pull-down, and immunoblot analysis. The immunoblot shows that Pin1-3 Pl engages Pin1 more potently than Pin1-3, while Pin1-3 P2 displays no engagement towards Pin1 under the conditions tested.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

A first aspect of the present invention is directed to a compound having a structure represented by formula (PIN1-3 P1):

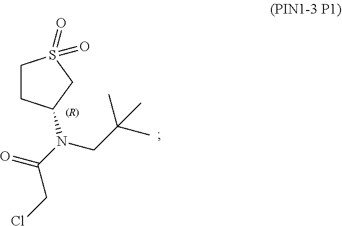

or a pharmaceutically acceptable salt thereof.

A further aspect of the present invention is directed to a compound having a structure represented by formula (PIN1-3 P2):

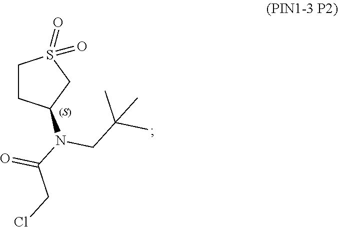

or a pharmaceutically acceptable salt thereof.

Another aspect of the present invention is directed to a pharmaceutical composition, comprising a therapeutically effective amount of any one of the compounds of formula PIN1-3 P1 or PIN1-3-P2, or a pharmaceutically acceptable salt thereof, and a pharmaceutically acceptable carrier. In some embodiments, the pharmaceutical composition is in the form of a solid. In some embodiments, the solid is in the form of a tablet or capsule. In some embodiments, the pharmaceutical composition is in the form of a liquid.

Another aspect of the present invention is directed to a method of treating a disease or disorder mediated by dysregulated Pin1 activity, comprising administering a therapeutically effective amount of the compound of formula PIN1-3 P1 or PIN1-3-P2, or a pharmaceutically acceptable salt thereof. In some embodiments, the disease is cancer. In some embodiments, the cancer is triple-negative breast cancer or MYCN-driven neuroblastoma.

In another aspect of the present invention, the method further comprises administering an immunotherapy. In some embodiments, the immunotherapy is a checkpoint inhibitor, a cell-cycle inhibitor, or a targeted therapy. In some embodiments, the checkpoint inhibitor is anti-PD-1 or anti-PD-L1. In some embodiments, the cell-cycle inhibitor is palbociclib, ribociclib, or abemaciclib. In some embodiments, the targeted therapy is a kinase inhibitor.

Another aspect of the present invention is directed to a method of reducing the activity of Pin1 in a cell, either in vivo or in vitro, comprising contacting the cell with the compound of formula PIN1-3 P1 or PIN1-3-P2, or a pharmaceutically acceptable salt thereof.

Another aspect of the present invention is directed to a method of making a compound of formula PIN1-3 P1:

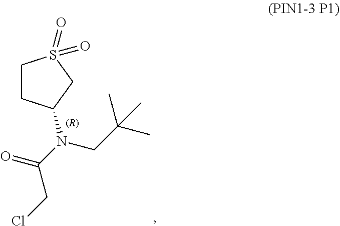

the method comprising:
a) forming a first reaction mixture comprising the compound of formula 1a:

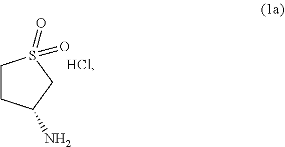

a non-nucleophilic base, a first solvent, and a compound of formula 3:

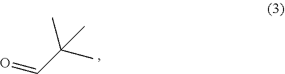

wherein the molar ratio of the compound of formula 1a to the compound of formula 3 is greater than 1.4, wherein the reaction mixture is mixed for at least 1.5 hours at or above room temperature under conditions suitable to form an imine;
wherein sodium triacetoxyborohydride (STAB) is added to the reaction mixture in one portion, wherein the molar ratio of STAB to the compound of formula 3 is greater than 2.0, wherein the reaction mixture is stirred for at least 12 hours at or above room temperature, thereby forming a compound of formula 2a:

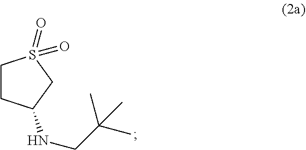

b) forming a second reaction mixture comprising a compound of formula 2a, a non-nucleophilic base, a second solvent, and a compound of formula 4:

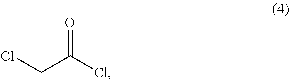

wherein the molar ratio of the compound of formula 4 to the compound of formula 2a is greater than 1.2, wherein the reaction mixture is mixed for at least 30 minutes at 0° C., thereby forming the compound of formula PIN1-3 P1.

Another aspect of the present invention is directed to a method of making a compound of formula PIN1-3 P2:

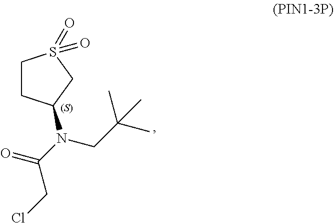

the method comprising:
a) forming a first reaction mixture comprising the compound of formula 1b:

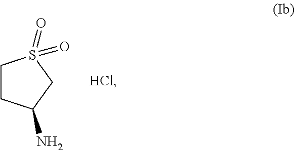

a non-nucleophilic base, a first solvent, and a compound of formula 3:

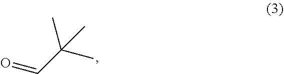

wherein the molar ratio of the compound of formula 1b to the compound of formula 3 is greater than 1.4, wherein the reaction mixture is mixed for at least 1.5 hours at or above room temperature under conditions suitable to form an imine;
wherein sodium triacetoxyborohydride (STAB) is added to the reaction mixture in one portion, wherein the molar ratio of STAB to the compound of formula 3 is greater than 2.0, wherein the reaction mixture is stirred for at least 12 hours at or above room temperature, thereby forming a compound of formula 2b:

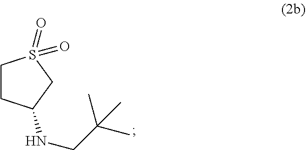

b) forming a second reaction mixture comprising a compound of formula 2b, a non-nucleophilic base, a second solvent, and a compound of formula 4:

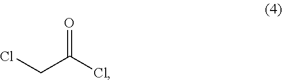

wherein the molar ratio of the compound of formula 4 to the compound of formula 2b is greater than 1.2, wherein the reaction mixture is mixed for at least 30 minutes at 0° C., thereby forming the compound of formula PIN1-3 P1. In some embodiments, the non-nucleophilic base is triethylamine. In some embodiments, the first solvent is dichloromethane. In some embodiments, the second solvent is acetonitrile.

In some embodiments, the use of PIN1-3-P1 results in improved efficacy of PIN1-3. In some embodiments, the use of PIN1-3-P1 results in reduced off-target toxicity effects of PIN1-3.

Compounds of the present invention may be in the form of a free acid or free base, or a pharmaceutically acceptable salt. As used herein, the term “pharmaceutically acceptable” refers to a material, such as a carrier or diluent, which does not abrogate the biological activity or properties of the compound, and is relatively non-toxic, i.e., the material may be administered to a subject without causing undesirable biological effects (such as dizziness or gastric upset) or interacting in a deleterious manner with any of the components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

In some embodiments, the compound of the present application is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

Compounds of the present invention may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present application may be made and used in the form of individual isomers and substantially free of other isomers, or in the form of a mixture of various isomers, e.g., racemic mixtures of stereoisomers.

In addition, the compounds of the present invention embrace the use of N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

### Methods of Synthesis

In another aspect, the present invention is directed to a method for making a compound of formula (PIN1-3 P1) or formula (PIN1-3 P2), or a pharmaceutically acceptable salt thereof. Broadly, the inventive compounds or pharmaceutically acceptable salts thereof may be prepared by any process known to be applicable to the preparation of chemically related compounds. The compounds of the present invention will be better understood in connection with the synthetic schemes that described in various working examples and which illustrate non-limiting methods by which the compounds of the invention may be prepared. In some embodiments, the inventive compounds are prepared using chiral HPLC to separate enantiomers from a racemic mixture.

### Pharmaceutical Compositions

Another aspect of the present invention is directed to a pharmaceutical composition that includes a therapeutically effective amount of the compound of formula (PIN1-3 P1) or formula (PIN1-3 P2) or a pharmaceutically acceptable salt thereof, and a pharmaceutically acceptable carrier. The term “pharmaceutically acceptable carrier,” as known in the art, refers to a pharmaceutically acceptable material, composition, or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may include one or more pharmaceutically acceptable excipients.

Broadly, compounds of formula (PIN1-3 P1) or formula (PIN1-3 P2) may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compositions are formulated for oral or intravenous administration (e.g., systemic intravenous injection).

Accordingly, compounds of the present invention may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate, or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of the present invention may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups, and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include excipients such as wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished using a liquid suspension or crystalline or amorphous material with poor water solubility. Prolonged absorption of the compound from a parenterally administered formulation may also be accomplished by suspending the compound in an oily vehicle.

In certain embodiments, compounds of formula (PIN1-3 P1) or formula (PIN1-3 P2) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The inventive compounds may be formulated for buccal or sublingual administration, examples of which include tablets, lozenges, and gels.

The compounds may be formulated for administration by inhalation. Various forms suitable for administration by inhalation include aerosols, mists, or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of formula (PIN1-3 P1) or formula (PIN1-3 P2) may be formulated for topical administration which as used herein, refers to administration intradermally by application of the formulation to the epidermis. These types of compositions are typically in the form of ointments, pastes, creams, lotions, gels, solutions, and sprays.

Representative examples of carriers useful in formulating compositions for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents can transport a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerine, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a compound of formula (PIN1-3 P1) or formula (PIN1-3 P2) or a pharmaceutically acceptable salt or a stereoisomer thereof; or a composition including the compound of formula (PIN1-3 P1) or formula (PIN1-3 P2) or a pharmaceutically acceptable salt or a stereoisomer thereof, effective in producing the desired therapeutic response in a particular patient suffering from a Pin1-mediated disease or disorder. The term “therapeutically effective amount” includes the amount of the compound of the application or a pharmaceutically acceptable salt or a stereoisomer thereof, when administered, may induce a positive modification in the disease or disorder to be treated (e.g., remission), or is sufficient to prevent development or progression of the disease or disorder, or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed and the particular pharmaceutically acceptable carrier utilized.

The total daily dosage of the compounds and usage thereof may be decided in accordance with standard medical practice, e.g., by the attending physician using sound medical judgment. The specific therapeutically effective dose for any particular subject will depend upon a variety of factors including the disease or disorder being treated and the severity thereof (e.g., its present status); the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, 10th Edition, A. Gilman, J. Hardman and L. Limbird, eds., McGraw-Hill Press, 155-173, 2001).

Compounds of the present invention and their pharmaceutically acceptable salts may be effective over a wide dosage range. In some embodiments, the total daily dosage (e.g., for adult humans) may range from about 0.001 to about 1000 mg, from 0.01 to about 1000 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosage may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present invention is directed to methods of treating diseases or disorders involving dysfunctional (e.g., dysregulated) Pin1 activity, that entails administration of a therapeutically effective amount of a compound of formula (PIN1-3 P1) or formula (PIN1-3 P2) or a pharmaceutically acceptable salt thereof, to a subject in need thereof.

The diseases or disorders may be said to be characterized or mediated by dysregulated or dysfunctional Pin1 activity (e.g., elevated levels of Pin1 relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health. In some embodiments, compounds of the application may be useful in the treatment of proliferative diseases and disorders (e.g., cancer or benign neoplasms). As used herein, the term “cell proliferative disease or disorder” refers to the conditions characterized by unregulated or abnormal cell growth, or both. Cell proliferative disorders include noncancerous conditions, precancerous conditions, and cancer.

Pin1-catalyzed prolyl isomerization regulates the functions of its substrates through multiple different mechanisms, including controlling catalytic activity, turnover, phosphorylation, interactions with DNA, RNA or other proteins, and subcellular localization and processing. Pin1 is tightly regulated normally and its deregulation can have a major impact on the development and treatment of cancer and neurodegenerative diseases.

Pin1 substrates comprise proteins involved in signal transduction, including RAF1, HER2, eNOS, SMAD2/3, Notch1, Notch3, AKT, FAK, P70S6K, PTP-PEST, MEK1, GRK2, CDK10, FBXW7, PIP4Ks, PKM2 and JNK1; proteins involved in gene transcription including SIN3-RPD3, JUN, β-catenin, CF-2, hSPT5, MYC, NF-κB, FOS, RARα, SRC-3/AIB1, STAT3, MYB, SMRT, FOXO4, KSRP, SF-1, Nanog, PML, Mutant p53, ΔNp63, Oct4, ERα, PKM2, AR, SUV39H1, RUNX3, KLF10, Osterix and PML-RARα; proteins involved in cell cycle at the G1/S including Cyclin D1, KI67, Cyclin E, p27, LSF and RB1; proteins involved in cell cycle at the G2/M and M including NIMA, RAB4, CDC25, WEE1, PLK1, MYT1, CDC27, CENP-F, INCENP, RPB1, NHERF-1, KRMP1, CK2, TOPIIa, DAB2, p54NRB, SIL, EMI1, CEP55, BORA, Survivin, SEPT9, SP1, SWI6, WHI5 and Separase; proteins involved in DNA damage/stress response and apoptosis including p53, BCL-2, p73, BIMEL, p66SHC, DAXX, MCL-1, NUR77, HIPK2, RBBP8, p63, HSF1, HIF-1α, CHE-1 and PGK1; proteins involved in immune response including NFAT, AUF1, IRF3, BTK, BAX, COX-2, p47PHOX, IRAK1, GR and FADD; proteins involved in viral or parasitic infection and transformation including HBX, A3G, v-Rel, Tax, Capsid protein, Integrase, BALF5, RTA, FBXW7 and ORF1p; proteins involved in neuronal survival and degeneration including TAU, APP, Synphilin-1, Gephyrin, mGluR5, REST, GRO/TLE1 and CRMP2A. (Zhou and Lu, “The isomerase Pin1 controls numerous cancer-driving pathways and is a unique drug target” Nature Reviews Cancer 16:463-478; Supplementary Information (2016)).

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

In general, methods of using the compounds of the present invention include administering to a subject in need thereof a therapeutically effective amount of a compound of the present invention.

Exemplary types of non-cancerous diseases or disorders that may be amenable to treatment with the compounds of the present invention include inflammatory diseases and conditions, autoimmune diseases, neurodegenerative diseases, heart diseases, viral diseases, chronic and acute kidney diseases or injuries, obesity, metabolic diseases, allergic and genetic diseases.

Representative examples of specific non-cancerous diseases and disorders include rheumatoid arthritis, alopecia areata, lymphoproliferative conditions, autoimmune hematological disorders (e.g. hemolytic anemia, aplastic anemia, anhidrotic ecodermal dysplasia, pure red cell anemia and idiopathic thrombocytopenia), cholecystitis, acromegaly, rheumatoid spondylitis, osteoarthritis, gout, scleroderma, sepsis, septic shock, dacryoadenitis, cryopyrin associated periodic syndrome (CAPS), endotoxic shock, endometritis, gram-negative sepsis, keratoconjunctivitis sicca, toxic shock syndrome, asthma, adult respiratory distress syndrome, chronic obstructive pulmonary disease, chronic pulmonary inflammation, chronic graft rejection, hidradenitis suppurativa, inflammatory bowel disease, Crohn's disease, Behcet's syndrome, systemic lupus erythematosus, glomerulonephritis, multiple sclerosis, juvenile-onset diabetes, autoimmune uveoretinitis, autoimmune vasculitis, thyroiditis, Addison's disease, lichen planus, appendicitis, bullous pemphigus, pemphigus vulgaris, pemphigus foliaceus, paraneoplastic pemphigus, myasthenia gravis, immunoglobulin A nephropathy, autoimmune thyroiditis or Hashimoto's disease, Sjogren's syndrome, vitiligo, Wegener granulomatosis, granulomatous orchitis, autoimmune oophoritis, sarcoidosis, rheumatic carditis, ankylosing spondylitis, Grave's disease, autoimmune thrombocytopenia purpura, psoriasis, psoriatic arthritis, eczema, dermatitis herpetiformis, ulcerative colitis, pancreatic fibrosis, hepatitis, hepatic fibrosis, CD14 mediated sepsis, non-CD14 mediated sepsis, acute and chronic renal disease, irritable bowel syndrome, pyresis, restenosis, cerebral malaria, cervicitis, stroke and ischemic injury, neural trauma, acute and chronic pain, allergic rhinitis, allergic conjunctivitis, chronic heart failure, congestive heart failure, acute coronary syndrome, cachexia, malaria, leprosy, leishmaniasis, Lyme disease, Reiter's syndrome, acute synovitis, muscle degeneration, bursitis, tendonitis, tenosynovitis, herniated, ruptured, or prolapsed intervertebral disk syndrome, osteopetrosis, rhinosinusitis, thrombosis, silicosis, pulmonary sarcosis, bone resorption diseases, such as osteoporosis, graft-versus-host reaction, fibromyalgia, AIDS and other viral diseases such as Herpes Zoster, Herpes Simplex I or II, influenza virus and cytomegalovirus, diabetes Type I and II, obesity, insulin resistance and diabetic retinopathy, 22q11.2 deletion syndrome, Angelman syndrome, Canavan disease, celiac disease, Charcot-Marie-Tooth disease, color blindness, Cri du chat, Down syndrome, cystic fibrosis, Duchenne muscular dystrophy, haemophilia, Klinefleter's syndrome, neurofibromatosis, phenylketonuria, Prader-Willi syndrome, sudden infant death syndrome, sickle cell disease, Tay-Sachs disease, Turner syndrome, urea cycle disorders, thalassemia, otitis, pancreatitis, parotitis, pericarditis, peritonitis, pharyngitis, pleuritis, phlebitis, pneumonitis, cystic fibrosis, uveitis, polymyositis, proctitis, interstitial lung fibrosis, dermatomyositis, arteriosclerosis, amyotrophic lateral sclerosis, asocality, immune response, varicosis, vaginitis, including chronic recurrent yeast vaginitis, depression, and Sudden Infant Death Syndrome.

In some embodiments, the autoimmune disease that is treated is lupus, asthma or arthritis.

In some embodiments, the neurodegenerative disease is Alzheimer's disease or Parkinson's disease.

In other embodiments, the methods are directed to treating subjects having cancer. Broadly, the compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) including leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., brain stem glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, acute promyelocytic leukemia, chronic lymphocytic leukemia, chronic myelogenous leukemia, chronic myeloproliferative disorders, anal cancer, colorectal cancer (e.g., colon cancer, rectal cancer), cutaneous T-cell lymphoma, lymphoid neoplasm, mycosis fungoids, Sézary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), germ cell tumor, ovarian germ cell tumor, gestational trophoblastic tumor glioma, head and neck cancer, hepatocellular (liver) cancer, Hodgkin's lymphoma, non-Hodgkin's lymphoma, hypopharyngeal cancer, intraocular melanoma, ocular cancer, islet cell tumors (endocrine pancreas), renal cancer (e.g., Wilm's Tumor, clear cell renal cell carcinoma), laryngeal cancer, acute lymphoblastic leukemia, acute myeloid leukemia, hairy cell leukemia, lip and oral cavity cancer, liver cancer, lung cancer (e.g., non-small cell lung cancer and small cell lung cancer), primary central nervous system lymphoma, Waldenstrom's macroglobulinema, melanoma, intraocular (eye) melanoma, merkel cell carcinoma, mesothelioma malignant, mesothelioma, metastatic squamous neck cancer, multiple endocrine neoplasia syndrome, mycosis fungoids, myelodysplastic syndromes, myelodyplastic/myeloproliferative diseases, multiple myeloma, chromic myeproliferative disorders, nasopharyngeal cancer, neuroblastoma, oral cancer (e.g., mouth cancer, lip cancer, oral cavity cancer, tongue cancer, oropharyngeal cancer, throat cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, retinoblastoma rhabdomyosarcoma, salivary gland cancer, uterine cancer (e.g., endometrial uterine cancer, uterine sarcoma, uterine corpus cancer), merkel cell skin carcinoma, squamous cell carcinoma, supratentorial primitive neuroectodermal tumors, testicular cancer, thymoma, thymoma and thymic carcinoma, thyroid cancer, transitional cell cancer of the renal pelvis and ureter and other urinary organs, urethral cancer, gestational trophoblastic tumor, vaginal cancer and vulvar cancer. In some embodiments, the cancer is triple-negative breast cancer or MYCN-driven neuroblastoma.

Sarcomas that may be treatable with compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue) and mesenchymous or mixed mesodermal tumor (mixed connective tissue types).

In some embodiments, methods of the present invention entail treatment of subjects having cell proliferative diseases or disorders of the hematological system, liver (hepatocellular), brain, lung, colorectal (e.g., colon), pancreas, prostate, ovary, breast, or skin (e.g., melanoma).

As used herein, “cell proliferative diseases or disorders of the hematologic system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), acute myeloid leukemia (AML), acute promyelocytic leukemia (APL), mantle cell lymphoma (MCL) and ALK+ anaplastic large cell lymphoma) (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, refractory B-cell non-Hodgkin's lymphoma, and relapsed B-cell non-Hodgkin's lymphoma), childhood lymphomas, and lymphomas of lymphocytic and cutaneous origin, e.g., small lymphocytic lymphoma, primary CNS lymphoma (PCNSL), marginal zone lymphoma (MZL), leukemia, including chronic lymphocytic leukemia (CLL), childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver (hepatocellular)” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the brain may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, a precancer or precancerous condition of the lung, benign growths or lesions of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer includes lung neoplasms having histologic and ultrastructural heterogeneity (e.g., mixed cell types).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer. Colon cancer includes malignant colon neoplasms, carcinoma in situ, typical carcinoid tumors, and atypical carcinoid tumors. Colon cancer includes adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polypopsis, Gardner's syndrome, Peutz-Jeghers syndrome, Turcot's syndrome and juvenile polyposis. Cell proliferative disorders of the colon can be characterized by hyperplasia, metaplasia, and dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, and dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell types).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the prostate.

The compounds of the present invention may be administered to a patient, e.g., a cancer patient, as a monotherapy or by way of combination therapy, and as a front-line therapy or a follow-on therapy for patients who are unresponsive to front line therapy. Therapy may be “first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which have been partially successful but are intolerant to the particular treatment. Therapy may also be given as an adjuvant treatment, i.e., to prevent reoccurrence of cancer in patients with no currently detectable disease or after surgical removal of a tumor. Thus, in some embodiments, the compound may be administered to a patient who has received another therapy, such as chemotherapy, radioimmunotherapy, surgical therapy, immunotherapy, radiation therapy, targeted therapy or any combination thereof. In some embodiments, the immunotherapy is a checkpoint inhibitor (e.g., anti-PD-1, anti-PD-L1), a cell-cycle inhibitor (e.g., palbociclib, ribociclib, abemaciclib), or a targeted therapy (e.g., kinase inhibitor).

The methods of the present invention may entail administration of compounds of the invention or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5 or 6 weeks, and in other embodiments entails a 28-day cycle which includes daily administration for 3 weeks (21 days).

### Combination Therapy

The compounds of the present invention may be used in combination with at least one other active agent, e.g., anti-cancer agent or regimen, in treating diseases and disorders. The term “in combination” in this context means that the agents are co-administered, which includes substantially contemporaneous administration, by the same or separate dosage forms, or sequentially, e.g., as part of the same treatment regimen or by way of successive treatment regimens. Thus, if given sequentially, at the onset of administration of the second compound, the first of the two compounds is, in some cases, still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a compound of the invention in combination with one or more additional therapeutics. The dosage of the additional therapeutic may be the same or even lower than known or recommended doses. See, Hardman et al., eds., Goodman & Gilman's The Pharmacological Basis of Therapeutics, 10th ed., McGraw-Hill, New York, 2001; Physician's Desk Reference 60th ed., 2006. Anti-cancer agents that may be used in combination with the inventive compounds are known in the art. See, e.g., U.S. Pat. No. 9,101,622 (Section 5.2 thereof). Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy. In some embodiments, the treatment regimen may include immunotherapy, In some embodiments, the immunotherapy is a checkpoint inhibitor (e.g., anti-PD-1, anti-PD-L1), a cell-cycle inhibitor (e.g., palbociclib, ribociclib, abemaciclib), or a targeted therapy (e.g., kinase inhibitor).

In some embodiments, the compound of formula (PIN1-3 P1) or formula (PIN1-3 P2) and the additional anticancer therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart. The two or more anticancer therapeutics may be administered within the same patient visit.

In some embodiments, the compound of formula (PIN1-3 P1) or formula (PIN1-3 P2) and the additional agent or therapeutic (e.g., an anti-cancer therapeutic) are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

The compounds of the present invention may be administered to a patient suffering from a neurodegenerative disease or disorder in combination with another active agent. Representative examples of other active agents known to treat neurodegenerative diseases and disorders include dopaminergic treatments (e.g., Carbidopa-levodopa, pramipexole (Mirapex), ropinirole (Requip) and rotigotine (Neupro, given as a patch)). Apomorphine and monoamine oxidase B (MAO-B) inhibitors (e.g., selegiline (Eldepryl, Zelapar), rasagiline (Azilect) and safinamide (Xadago)) for Parkinson disease and movement disorders, cholinesterase inhibitors for cognitive disorders (e.g., benztropine (Cogentin) or trihexyphenidyl), antipsychotic drugs for behavioral and psychological symptoms of dementia, as well as agents aimed to slow the development of diseases, such as Riluzole for ALS, cerebellar ataxia and Huntington's disease, non-steroidal anti-inflammatory drugs for Alzheimer's disease, and caffeine A2A receptor antagonists and CERE-120 (adeno-associated virus serotype 2-neurturin) for the neuroprotection of Parkinson's disease.

The compounds of the present invention may be administered to a patient suffering from an autoimmune disease or disorder in combination with another active agent. Representative examples of other active agents known to treat neurodegenerative diseases and disorders include corticosteroids (e.g., prednisone, hydrocortisone, and dexamethasone) immunosuppressant drugs, such as methotrexate, cyclophosphamide, and azathioprine. Other examples include immunosuppressive dugbelimumab (Benlysta®) for severe active lupus nephritis or severe active central nervous system lupus, asthma and arthritis, anti-malarial dugs (e.g., hydroxychloroquine (Plaquenil®) and chloroquine (Aralen®)) for lupus, combinations of corticosteroid and bronchodilator (e.g., fluticasone and salmeterol (Advair Diskus®), budesonide and formoterol (Symbicort®), and fluticasone and vilanterol (BREO)), and analgesics (e.g., acetaminophen), nonsteroidal anti-inflammation drugs (NSAIDs) (e.g., aspirin, ibuprofen, naproxen, indomethacin and celecoxib (Celebrex®)), traditional disease-modifying antirheumatic drugs (DMARDs) (e.g., tumor necrosis factor (TNF) inhibitors or TNF blockers (etanercept (Enbrel®) and adalimumab (Humira®)), Interleukin-6 (IL-6) inhibitors, Interleukin-1 (IL-1) receptor antagonists, B-cell inhibitors, Janus kinases (JAK) inhibitors, phosphodiesterase 4 (PDE 4) inhibitors and costimulation modulators) for treating rheumatoid arthritis (RA), ankylosing spondylitis, psoriatic arthritis, juvenile idiopathic arthritis and lupus.

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain the compound of the present application or a pharmaceutical composition. The kits or pharmaceutical systems of the invention may also include printed instructions for using the compounds and compositions.

These and other aspects of the present application will be further appreciated upon consideration of the following Examples, which are intended to illustrate certain particular embodiments of the application but are not intended to limit its scope, as defined by the claims.

### EXAMPLES

Broadly, the inventive compounds or pharmaceutically acceptable salts thereof, may be prepared by any process known to be applicable to the preparation of chemically related compounds, including, but not limited to, separation using chiral HPLC. Representative schemes for synthesizing the compounds of the present invention are described below.

### Example 1: Synthesis of Pin1-3 P1

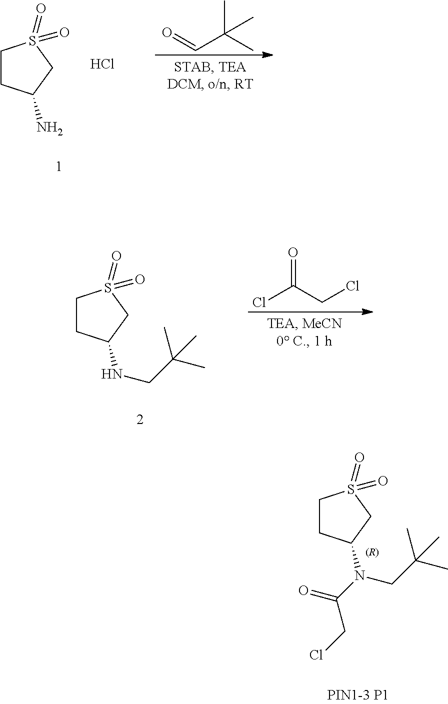

((R)-3-(neopentylamino)tetrahydrothiophene 1,1-dioxide): To a mixture of (R)-3-aminotetrahydrothiophene 1,1-dioxide hydrochloride (100 mg, 0.58 mmol) and pivalaldehyde (34 mg, 0.39 mmol) in dichloromethane (DCM) (4 mL) was added triethylamine (TEA) (103 μL, 0.58 mmol). The reaction mixture was stirred at room temperature for 2 hours. Subsequently, sodium triacetoxyborohydride (STAB) (165 mg, 0.78 mmol) was added in one portion and the reaction was stirred overnight. The reaction mixture was concentrated in vacuo and the crude residue was purified by column chromatography on silica gel (0-20% MeOH in 1.75 N NH₃/DCM) to obtain (R)-3-(neopentylamino)tetrahydrothiophene 1,1-dioxide (2) as an off-white solid (75 mg, 0.37 mmol, 94% yield). ¹H NMR (500 MHZ, Chloroform-d) δ 3.62-3.57 (m, 1H), 3.36-3.29 (m, 2H), 3.10-3.05 (m, 1H), 2.90 (dd, J=13.2, 6.3 Hz, 1H), 2.47-2.39 (m, 1H), 2.36 (d, J=5.2 Hz, 1H), 2.12 (s, 3H), 0.93 (s, 9H). MS m/z 206.32 [M+H]⁺.

((R)-2-chloro-N-(1,1-dioxidotetrahydrothiophen-3-yl)-N-neopentylacetamide):

Intermediate 2 (40 mg, 0.19 mmol) was added in a mixture of acetonitrile (MeCN) (1 mL) and triethylamine (80 μL, 0.57 mmol). The reaction mixture was cooled to 0° C., followed by dropwise addition of 1M solution of 2-chloroacetyl chloride in DCM (230 μL, 0.23 mmol). The reaction mixture was stirred for 1 hour at 0° C.and purified by reverse phase HPLC (20-80% MeOH in H₂O) to obtain ((R)-2-chloro-N-(1,1-dioxidotetrahydrothiophen-3-yl)-N-neopentylacetamide (Pin1-3 P1) as a yellow oil (4.4 mg, 0.011 mmol, 12% yield). ¹H NMR (500 MHZ, DMSO-d6) δ 4.39-4.32 (m, 2H), 4.02-3.95 (m, 1H), 3.48 (m, 2H), 3.33 (m, 1H), 3.27-3.19 (m, 2H), 3.11 (m, 1H), 2.39 (m, 3H), 0.94 (s, 9H). MS m/z 282.32 [M+H]+.

### Example 2: Syntheses of Pin1-3 P2

((S)-2-chloro-N-(1,1-dioxidotetrahydrothiophen-3-yl)-N-neopentylacetamide):

Following the general procedure described for Pin1-3 P1, (S)-3-aminotetrahydrothiophene 1,1-dioxide was used to synthesize (S)-2-chloro-N-(1,1-dioxidotetrahydrothiophen-3-yl)-N-neopentylacetamide (Pin1-3 P2) as a yellow oil. ¹H NMR (500 MHZ, DMSO-d6) δ 4.39-4.32 (m, 2H), 4.04-3.95 (m, 1H), 3.48 (m, 2H), 3.34 (m, 1H), 3.24 (m, 2H), 3.14-3.08 (m, 1H), 2.39 (m, 2H), 0.94 (s, 9H). MS m/z 282.28 [M+H]⁺.

### Example 3: Enantiomer Excess Evaluation of Pin1-3 P1

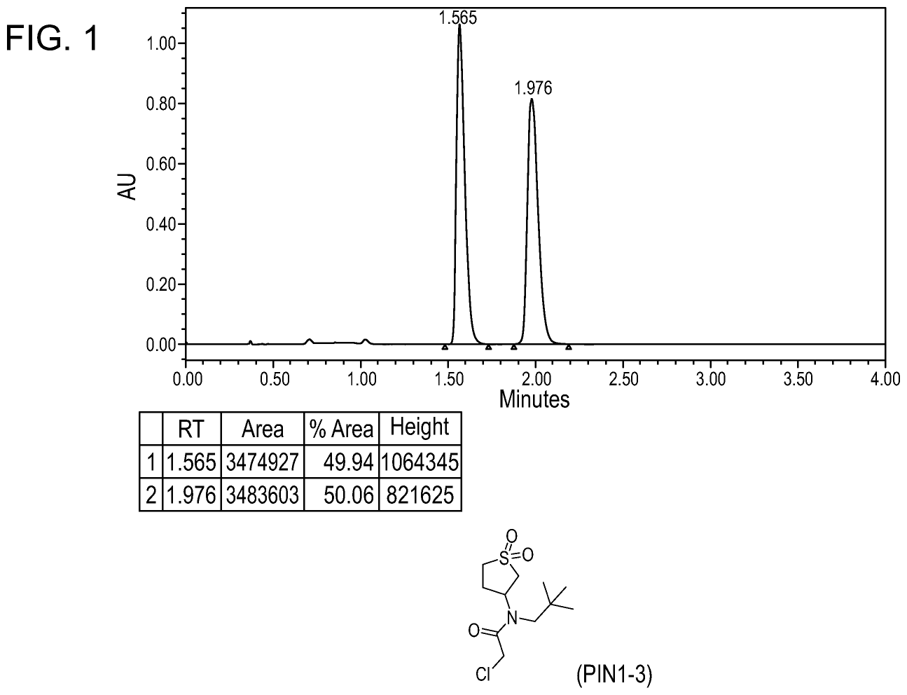

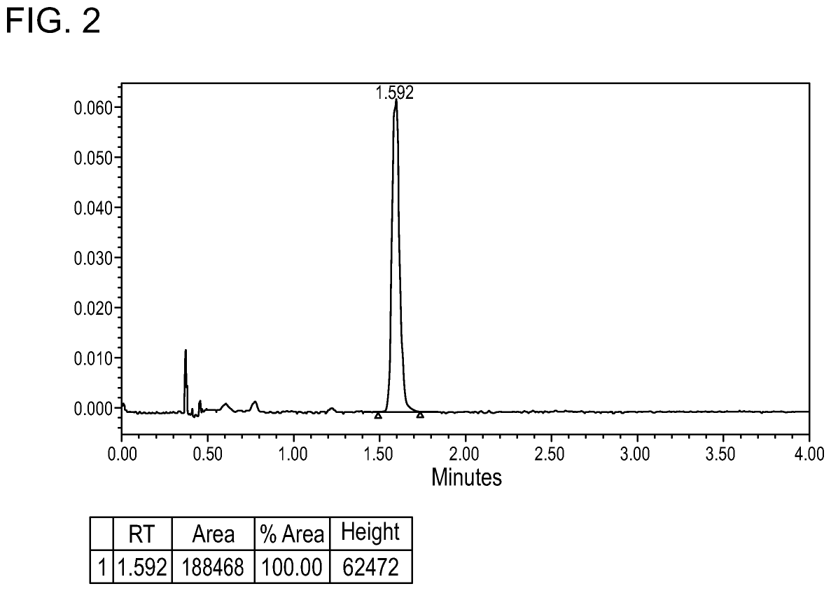

Chiral HPLC was performed on Pin1-3. Results, illustrated in FIG. 1, indicate that Pin1-3 consists of its two enantiomers in a 1:1 ratio. The first-eluting enantiomer, at retention time 1.565 min, displayed 49.94 area %, while the second-eluting enantiomer, at retention time 1.976 min, displayed 50.06 area %. Chiral HPLC was then performed on Pin1-3 P1. Results, illustrated in FIG. 2, indicate that Pin1-3 P1 displays an enantiomeric excess (e.e.) of >99%. The Pin1-3 P1 retention time matches that of the first-eluting enantiomer from the chiral HPLC performed for Pin1-3.

### Example 4: Fluorescence Polarization (FP) (binding to Pin1)

Binding affinity to Pin1 was determined using a fluorescence polarization assay. Kᵢ values obtained from the FP assay results were derived from the Kenakin Kᵢ equation: Kenakin Kᵢ=(Lb)(EC₅₀)(K_{d})/(Lo)(Ro)+Lb(Ro−Lo+Lb−K_{d}), where K_{d} [M]: K_{d} of the probe, EC₅₀ [M]: obtained from FP assay, total tracer Lo [M]: probe concentration in FP, bound tracer Lb [M]: 85% of probe concentration binds to target protein, total receptor Ro [M]: Pin1 concentration in the FP assay, as described (Auld et al. Receptor binding assays for HTS and drug discovery. in Assay Guidance Manual eds. Sittampalam, G.S., et al. Eli Lilly & Company and the National Center for Advancing Translational Sciences, 2004).

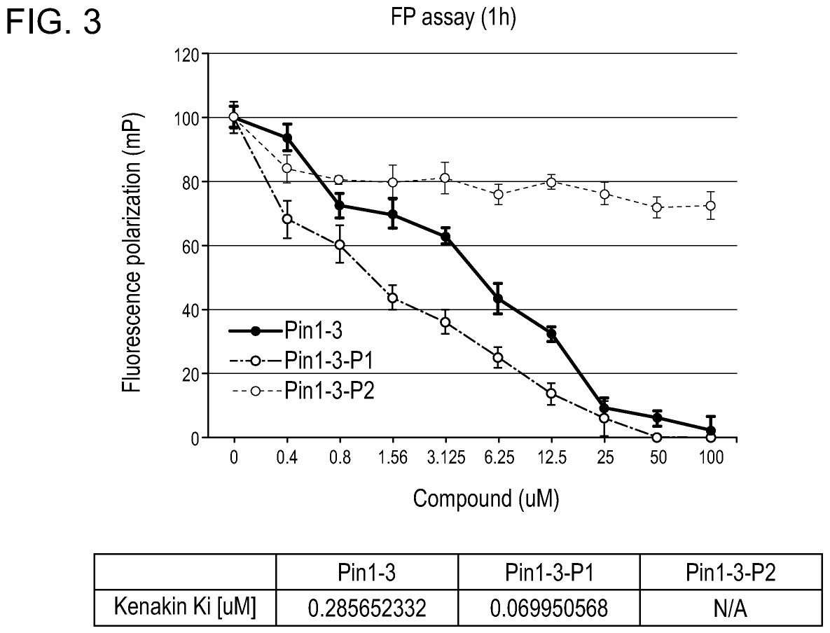

Results, illustrated in FIG. 3, show that compound Pin1-3 displays a Kᵢ of about 286 nM, Pin1-3 P1 displays a Kᵢ of about 70.0 nM. A Kᵢ was unable to be determined for Pin1-3 P2 under the conditions tested.

### Example 5: Treatment of Molt4 Cells

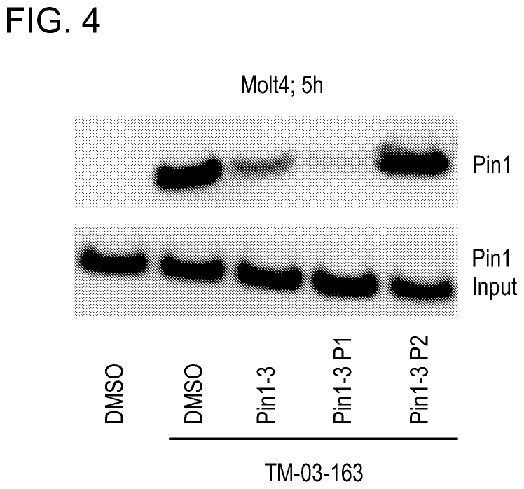

Molt4 cells were treated in a competition format with Pin1-3, Pin1-3 P1, and Pin1-3 P2 for 5 h, followed by cell lysis, incubation with TM-03-13 (Pin1-3 desthiobiotin), followed by streptavidin pulldown and immunoblot analysis. Results, illustrated in FIG. 4, show that Pin1-3 P1 engages Pin1 more potently than Pin1-3, while Pin1-3 P2 did not display engagement under the conditions tested.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A compound having a structure represented by formula (PIN1-3 P1) or (PIN1-3 P2): 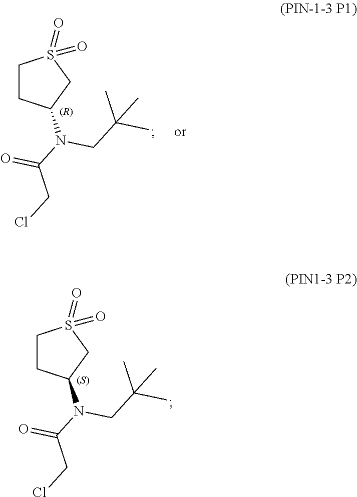
or a pharmaceutically acceptable salt thereof.

2. (canceled)

3. A pharmaceutical composition, comprising a therapeutically effective amount of the compound of claim 1, or a pharmaceutically acceptable salt thereof, and a pharmaceutically acceptable carrier.

4. The pharmaceutical composition of claim 3, which is in the form of a solid.

5. The pharmaceutical composition of claim 4, which is in the form of a tablet or capsule.

6. The pharmaceutical composition of claim 3, which is in the form of a liquid.

7. A method of treating a disease or disorder mediated by dysregulated Pin1 activity, comprising administering a therapeutically effective amount of the compound or pharmaceutically acceptable salt of claim 1.

8. The method of claim 7, wherein the disease is cancer.

9. The method of claim 8, wherein the cancer is triple-negative breast cancer or MYCN-driven neuroblastoma.

10. The method of claim 7, further comprising administering an immunotherapy.

11. The method of claim 10, wherein the immunotherapy is a checkpoint inhibitor, a cell-cycle inhibitor, or a targeted therapy.

12. The method of claim 11, wherein the checkpoint inhibitor is anti-PD-1 or anti-PD-L1.

13. The method of claim 11, wherein the cell-cycle inhibitor is palbociclib, ribociclib, or abemaciclib.

14. The method of claim 11, wherein the targeted therapy is a kinase inhibitor.

15. A method of reducing the activity of Pinl in a cell, either in vivo or in vitro, comprising contacting the cell with the compound of claim 1.

16. A method of making a compound of formula PIN1-3 P1: 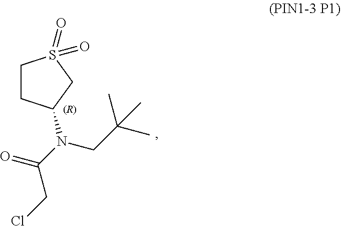
the method comprising:
a) forming a first reaction mixture comprising the compound of formula 1a: 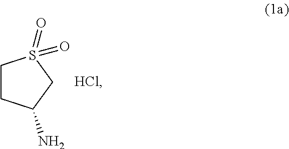
a non-nucleophilic base, a first solvent, and a compound of formula 3: 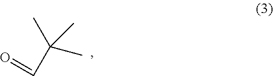
wherein the molar ratio of the compound of formula 1a to the compound of formula 3 is greater than 1.4, wherein the reaction mixture is mixed for at least 1.5 hours at or above room temperature under conditions suitable to form an imine;
wherein sodium triacetoxyborohydride (STAB) is added to the reaction mixture in one portion, wherein the molar ratio of STAB to the compound of formula 3 is greater than 2.0, wherein the reaction mixture is stirred for at least 12 hours at or above room temperature, thereby forming a compound of formula 2a: 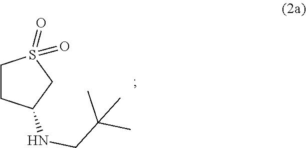
b) forming a second reaction mixture comprising a compound of formula 2a, a non-nucleophilic base, a second solvent, and a compound of formula 4: 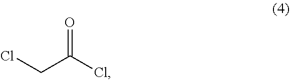
wherein the molar ratio of the compound of formula 4 to the compound of formula 2a is greater than 1.2, wherein the reaction mixture is mixed for at least 30 minutes at 0° C., thereby forming the compound of formula PIN1-3 P1, or
a method of making a compound of formula PIN1-3 P2: 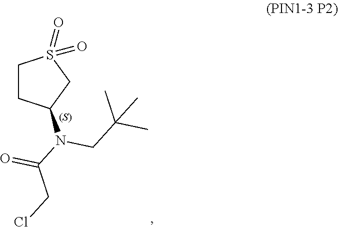
the method comprising:
a) forming a first reaction mixture comprising the compound of formula 1b: 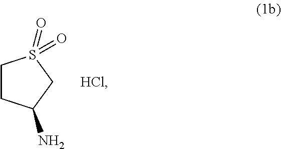
a non-nucleophilic base, a first solvent, and a compound of formula 3: 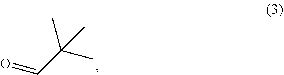
wherein the molar ratio of the compound of formula 1b to the compound of formula 3 is greater than 1.4, wherein the reaction mixture is mixed for at least 1.5 hours at or above room temperature under conditions suitable to form an imine:
wherein sodium triacetoxyborohydride (STAB) is added to the reaction mixture in one portion, wherein the molar ratio of STAB to the compound of formula 3 is greater than 2.0, wherein the reaction mixture is stirred for at least 12 hours at or above room temperature, thereby forming a compound of formula 2b: 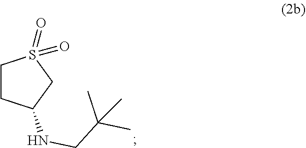
b) forming a second reaction mixture comprising a compound of formula 2b, a non-nucleophilic base, a second solvent, and a compound of formula 4: 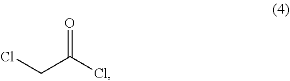
wherein the molar ratio of the compound of formula 4 to the compound of formula 2b is greater than 1.2. wherein the reaction mixture is mixed for at least 30 minutes at 0° C., thereby forming the compound of formula PIN1-3 P2.

17. (canceled)

18. The method of claim 16, wherein the non-nucleophilic base is triethylamine.

19. The method of claim 18, wherein the first solvent is dichloromethane.

20. The method of claim 18, wherein the second solvent is acetonitrile.
